(19) United States
(12) Patent Application Publication
(10) Pub. No.: US 2002/0146451 A1
(43) Pub. Date: Oct. 10, 2002

(54) Method for the administration of acid-labile drugs

(76) Inventors: Virender K. Sharma, Little Rock, AR (US); Colin W. Howden, Chicago, IL (US)

Correspondence Address: DAVIS CHIN, 16061 S. 94TH AVENUE, ORLAND HILLS, IL, 60477-4623, US

(21) Appl. No.: 09/824,847

(22) Filed: Apr. 4, 2001

Related U.S. Application Data
(60) Provisional application No. 60/218,509, filed on Jul. 15, 2000.

(51) Int. Cl. A61K 38/43; A61K 9/48; A61K 9/20; A61K 31/4184

(52) U.S. Cl. 424/452; 424/465; 424/94.1; 514/394

(57) ABSTRACT

A method for the formulation and delivery for administration of acid-labile drugs to human beings and other animals achieved by mixing the active pharmaceutical compound with a basic salt in a solution or suspension of any kind, where the basic salt solution or suspension protects the pharmaceutical compound from the adverse effects of gastric acid by neutralizing gastric acid.

### CROSS-REFERENCE TO RELATED APPLICATIONS

This application is continuation-in-part of provisional application Ser. No. 60/218,509 filed on Jul. 15, 2000.

### BACKGROUND OF THE INVENTION

1. Field of the Invention

The present invention relates to pharmaceutical preparations containing an acid-labile/drug, such as a substituted benzimidazole/proton pump inhibitor, PPI, or a preparation of pancreatic enzymes. More particularly, the present invention relates to a new method for administering such drugs either orally or by means of an artificial feeding tube, such as artificial tubes leading into the gastrointestinal tract including, but not limited to nasogastric, nasoduodenal, nasojejunal, orogastric, oroduodenal, orojejunal, gastrostomy and jejunostomy tubes. The gastrostomy and jejunostomy tubes may be created by any means known in the art such as surgically, radiologically or endoscopically.

2. Description of the Prior Art

Omeprazole, lansoprazole, pantoprazole, rabeprazole and esomeprazole are examples of substituted benzimidazoles, which are inhibitors of the proton pump, an enzyme also called H+/K+-ATPase, found on the surface of acid-secreting parietal cells in the stomach. The substituted benzimidazoles are referred to as proton pump inhibitors or PPI's. Typically, omeprazole and lansoprazole are administered as gelatin capsules containing enteric-coated granules of the drug. The gelatin capsule and enteric coating formulations are necessary to prevent naturally occurring gastric acid from denaturing these drugs, which are acid-labile. This formulation allows the drug, omeprazole or lansoprazole, to be absorbed into the circulatory system from the duodenum or proximal small intestine. Pantoprazole and rabeprazole are newer PPI's administered as entericcoated tablets also to prevent them from degradation by stomach acid. A purified from of the S-enantiomer of omeprazole is esomeprazole, which in the industry is expected to be the next PPI introduced into clinical practice. Other PPI's at early stages of development include leminoprazole.

PPI's are powerful inhibitors of gastric acid secretion. PPI's are used clinically to treat a variety of disorders related to excess acid secretion or the presence of stomach acid in abnormal sites, such as the esophagus. Therefore, PPI's are used to treat gastroesophageal reflux disease, GERD, whether or not it is associated with erosive esophagitis. The PPI's are also used to treat peptic ulceration occurring in the duodenum, stomach or other more unusual sites in the gastrointestinal tract. In combination with antibiotics, PPI's can be used to treat infection with a bacterium called Helicobacter pylori, H. pylori, which has been linked to inflammation of the stomach or gastritis. The Helicobacter pylori bacterium has also been associated with ulcers of the duodenum and stomach, gastric cancer and a type of gastric lymphoma.

Not all patients who might benefit from treatment with a PPI are able to swallow intact capsules or tablets. Patients having a condition leading to partial or complete obstruction of the esophagus or the area of entry of the esophagus into the stomach may have difficulty in swallowing, which is referred to as dysphagia. Other patients may have dysphagia due to neurological impairment from a variety of conditions, which may include cerebrovascular disease, a stroke, or dementia. Unconscious or critically ill patients may require PPI treatment, but they are unable to swallow intact capsules or tablets. A currently available PPI, pantoprazole, may be introduced through an intravenous formulation. Otherwise, there are only limited options for administering a PPI to patients who have difficulty swallowing. It is not possible to crush tablets of pantoprazole or rabeprazole for administration either orally or through a feeding tube.

It has been shown in the article entitled Nonencapsulated, Intact Omeprazole Granules Effectively Inhibit Intragastric Acidity When Administered Via a Gastrostomy, from American Journal of Gastroenterology 1997, Volume 92, pages 848 to 851, that intact omeprazole granules can be administered to human beings when the granules are suspended in orange juice and introduced via a gastrostomy tube. It was likewise noted in the article entitled The Pharmacodynamics of Lansoprazole Administered Via Gastrostomy as Intact, Non-Encapsulated Granules from the publication Alimentary Pharmacology and Therapeutics 1998, Volume 12, pages 1172 to 1174, that intact lansoprazole granules can also be administered via a gastrostomy tube to human beings when the granules are suspended in orange juice. Both omeprazole and lansoprazole produced the desired effect of suppressing gastric acid secretion in these experiments. This was similar to an expected effect from administering identical doses of these compounds as intact capsules to human beings.

Subsequently, it was noted in the article entitled The Effects on Intragastric Acidity of Pergastrostomy Administration of an Alkaline Suspension of Omeprazole, in Alimentary Pharmacology and Therapeutics 1999a, Volume 13 at pages 1091 to 1095, that omeprazole granules suspended in 8.4% sodium bicarbonate solution can also suppress gastric acid secretion when administered to human beings via a gastrostomy tube. However, the magnitude of the effect on acid secretion from the omeprazole-bicarbonate solution was less than that effect observed from either intact omeprazole capsules or intact omeprazole granules suspended in orange juice, which effects were reported in the above-cited study described in the above article from the American Journal of Gastroenterology. The suspension of omeprazole granules in 8.4% sodium bicarbonate solution has been termed simplified omeprazole suspension, SOS, and is described in U.S. Pat. No. 5,840,737 to Phillips which is hereby incorporated by reference in its entirety. The Phillips patent teaches an aqueous solution/suspension of substituted benzimidazoles in a carrier comprising a bicarbonate salt of a Group 1A metal.

Lansoprazole granules can be suspended in 8.4% sodium bicarbonate solution. The resultant suspension has been termed simplified lansoprazole suspension, SLS, and is also discussed in the aforementioned '737 patent to Phillips. SLS administered through a gastrostomy tube to human beings was noted as effective in inhibiting gastric acid secretion in the article entitled Simplified Lansoprazole Suspension—a Liquid Formulation of Lansoprazole—Effectively Suppresses Intragastric Acidity When Administered Through a Gastrostomy in the American Journal of Gastroenterology 1999b, Volume 94 at pages 1813 to 1817. The reported therapeutic effect of SLS was similar to the effect previously obtained with intact lansoprazole granules suspended in orange juice from the above-cited article in Alimentary Pharmacology and Therapeutics 1998, and that effect obtained using the same dose of lansoprazole administered to human beings as intact capsules, which is discussed in the article entitled The Effects of Oral Doses of Lansoprazole and Omeprazole on Gastric pH from the Journal of Clinical Gastroenterology 1997, Volume 24, pages 65 to 70.

There is a difference between the availability of omeprazole from SOS and of lansoprazole from SLS. Lansoprazole is well absorbed from SLS when given orally to human beings but omeprazole is not well absorbed when SOS is given orally to human beings. This difference was described in the article entitled Oral Pharmacokinetics of Omeprazole and Lansoprazole After Single and Repeated Doses as Intact Capsules and as Suspensions in Sodium Bicarbonate from Alimentary Pharmacology and Therapeutics 2000a, Volume 14, pages 887 to 892. The antisecretory effect of SLS has been noted as similar to that effect noted by other observers when intact lansoprazole capsules were given orally to human beings. However, the effect of SOS was less than the effect seen when intact omeprazole capsules were administered to a patient. These effects were variously described in the above-noted articles in American Journal of Gastroenterology 1999b, the Journal of Clinical Gastroenterology 1997, and the Alimentary Pharmacology and Therapeutics 1999a. In a recent randomized, single-dose crossover study in healthy human volunteers, SLS was found to be bioequivalent to a comparable dose of lansoprazole administered as an intact capsule, which was reported in the above-cited article from the American Journal of Gastroenterology 2000b.

SOS usage has been studied in critically ill patients for the prophylaxis of a potentially serious condition called stress-related gastric mucosal disease. This condition may cause bleeding from the stomach in patients who are critically ill for another reason. When stress-related gastric mucosal disease occurs, it can be serious or life threatening. Therefore, it is important to try to prevent this condition. High standards of care in Intensive Care Units, ICU, have helped to reduce the incidence of significant bleeding from stress-related mucosal disease. However, patients are still given drugs to try and prevent this complication. Drugs used in this context have included antacids, sucralfate and histamine H2-receptor antagonists, H2RAs. None of these drugs has produced a convincing benefit in terms of reducing the morbidity and/or mortality from stress-related mucosal bleeding in critically ill patients in ICU. PPIs may produce a more beneficial effect than the H2RAs since the former are associated with a greater degree of suppression of gastric acid secretion, which suppression is also more consistent and longer lasting.

Until recently, problems related to drug administration made it impossible to study the effects of PPIs in an ICU setting. Patients in ICU are typically unable to take capsules or tablets by mouth. In a small study, ICU patients received two 40 mg doses of SOS on the first day of the study, and a single 20 mg daily dose on subsequent days. This was reported in an article entitled A Prospective Study of Simplified Omeprazole Suspension for the Prophylaxis of Stress-Related Mucosal Damage in Critical Care Medicine 1996, Volume 24, pages 1793 to 1800. In this uncontrolled study, no bleeding from stress-related gastric mucosal disease was found in patients receiving SOS.

Other suspensions of omeprazole have been previously described in the following respective articles entitled: Development of an Oral Formulation of Omeprazole from Gastroenterology 1985, Volume 108 at pages 113 to 120; The Pharmacokinetics of Omeprazole in Humans—a Study of Single and Intravenous and Oral Doses from Therapeutic Drug Monitoring 1990, Volume 12 at pages 163 to 172; Pharmacokinetics and Bioavailability of Omeprazole After Single and Repeated Oral Administration in Healthy Subjects, from British Journal of Clinical Pharmacology 1990, Volume 29 at pages 557 to 563; Pharmacokinetic Study of Omeprazole in Elderly Healthy Volunteers from Clinical Pharmacokinetics 1992, Volume 23 at pages 469 to 476; and, Pharmacokinetics of [14C]-omeprazole in patients with liver cirrhosis, Clinical Pharmacokinetics 1993; 24: 71-78. The reported studies administered omeprazole with large volumes of sodium bicarbonate solution by mouth to healthy human volunteers, which administration was usually for the purpose of conducting experiments on the drug's pharmacokinetics. These formulations are unsuitable for clinical use due to the requisite large volumes of liquid and, the large sodium and bicarbonate content.

Omeprazole has also been formulated as a mixture in polyethylene glycols formed in a mixture of adeps solidus and sodium lauryl sulfate in a soluble, basic amino acid to produce a formulation designed for rectal administration as taught in U.S. Pat. No. 5,219,870 to Kim.

U.S. Pat. No. 5,395,323 to Berglund discloses a device for mixing a pharmaceutical from a solid supply into a parenterally acceptable liquid form for parenteral administration to human beings. This pharmaceutical administration focuses on the use of an omeprazole tablet placed in a device, dissolved with normal saline solution and infused into a patient. This device and method for infusion do not administer the omeprazole enterally, and do not deliver the omeprazole directly to its site of action, specifically the upper gastrointestinal tract.

U.S. Pat. No. 4,786,505 to Lovgren et al provides a pharmaceutical preparation consisting of omeprazole and an alkaline-reacting compound or an alkaline salt of omeprazole optionally together with an alkaline compound as a core material in a tablet formulation. The use of the alkaline material, which can be chosen from among several substances such as the sodium salt of carbonic acid, are used to form a micro-pH around each omeprazole particle to protect the omeprazole, which is highly sensitive to acid. The powder mixture is then formulated into small beads, pellets or tablets, which may be loaded into capsules by conventional pharmaceutical procedures. This formulation or form of omeprazole does not allow for the oral or tube administration of the drug to critically ill patients, patients unable to swallow normally, or non-critically ill patients requiring tube administration.

An alternative suspension of lansoprazole has also been described whereby lansoprazole granules are suspended in a flavoring solution. This description was noted in an article entitled A Lansopazole Suspension Formulation as an Alternative to Capsules for Oral Administration from Digestion 1998, Volume 59 at page 226. No clinical studies have been reported with this suspension apart from a single study of lansoprazole's absorption pharmacokinetics when this lansoprazole suspension was given by mouth to healthy human volunteers, which was noted in the article from Digestion 1998. It is not presently known if this suspension formulation has any clinical potential.

Lansoprazole has also been administered to human beings as intact granules mixed in applesauce for oral administration, as well as intact granules suspended in apple juice for administration via a nasogastric tube. The results from these administration techniques are noted in articles entitled Lansoprazole: An Alternative Method of Administration of a Capsule Dosage Formulationin Clinical Therapeutics 1995, Volume 17 at pages 441 to 447, and Lansoprazole: Administration of the Contents of a Capsule Dosage Formulation Through a Nasogastric Tubein Clinical Therapeutics 1996 in Volume 18 at pages 833 to 842. Neither of these administration techniques or formulations would be appropriate for administering to patients who were unconscious or critically ill. The Food and Drug Administration, or FDA, has also approved the administration of lansoprazole granules in a variety of fruit juices as well as in strained pears, yogurt and Ensure™ pudding.

In an article entitled A Prospective Study of Omeprazole Suspension to Prevent Clinically Significant Gastrointestinal Bleeding from Stress Ulcers in Mechanically Ventilated Trauma Patients from The Journal of Trauma: Injury, Infection, and Critical Care, Volume 44(3), March 1998, pages 527 to 533, the results were reported for the administration of simplified omeprazole suspension, SOS, to mechanically ventilated trauma patients at high risk for stress ulcers. This study reported that reliable administration of omeprazole or lansoprazole could not be consistently accomplished by instillation of the intact granules in juice. Further, it noted that enteric-coated granules are very adhesive when wet, which required flushing 6-10 granules at a time through a nasogastric tube with water. This flushing was considered to use excessive water, and the technique was considered to be impractical. The subsequent work led to the development or evolution of SOS, which has an enteric coating that is soluble in bicarbonate. It was noted that pH control from the usage of SOS is more reliable than when intact granules are used for the introduction of the medication.

In view of the above-described problems, it would therefore be desirable to provide a new and novel method for administering an acid-labile drug, such as a substituted benzimidazole/proton pump inhibitor, PPI, or a preparation of pancreatic enzymes, either orally or by means of an artificial feeding tube, to patients who are unable to swallow intact capsules or tablets. It would also be expedient to provide an acid-labile pharmaceutical compound having at least substituted benzimidazoles and pancreatic enzymes supplements which can be administered to patients unable to swallow intact capsules or tablets for neutralization of gastric acid and/or temporary stimulation of gastric acid secretion.

### SUMMARY OF THE INVENTION

Accordingly, it is a general object of the present invention to provide an acid-labile pharmaceutical compound having at least substituted benzimidazoles and pancreatic enzymes supplements and a method for administering the same which has been traditionally unavailable.

It is an object of the present invention to provide an improved method for administering an acid-labile drug, such as a substituted benzimidazole/proton pump inhibitor, PPI, or a preparation of pancreatic enzymes, either orally or by means of an artificial feeding tube, to patients who are unable to swallow intact capsules or tablets.

It is another object of the present invention to provide an acid-labile pharmaceutical compound having at least substituted benzimidazoles and pancreatic enzymes supplements which can be administered to patients unable to swallow intact capsules or tablets for neutralization of gastric acid and/or temporary stimulation of gastric acid secretion.

It is still another object of the present invention to provide an acid-labile pharmaceutical compound having at least substituted benzimidazoles including granules of omeprazole, granules of lansoprazole, tablets of pantoprazole, and tablets of rabeprazole suspended in one of calcium carbonate, magnesium hydroxide, and aluminum hydroxide suspension for oral administration to human beings.

In one aspect of the present invention, there is provided a pharmaceutical composition, which may include an aqueous suspension of an acid-labile drug such as substituted benzimidazoles/PPIs and pancreatic enzyme supplements, in a pharmaceutically acceptable carrier. The carrier may include a solution or suspension of the carbonate, bicarbonate or hydroxide salt of a metal, which metal salt may be selected from among the salts of calcium, magnesium and aluminum, but the carrier is not limited to such specific metal salts. A pharmaceutical composition of a solid mixture of an acid-labile drug is also taught and may include, but is not limited to, substituted benzimidazoles/PPIs and pancreatic enzyme supplements having a solid-phase basic salt of a metal formulated as a capsule, a standard tablet or an effervescent tablet. This latter basic metal salt may include, but is not limited to, salts of sodium, potassium, calcium, magnesium and aluminum.

For substituted benzimidazoles/PPIs, in another aspect of the present invention there is also provided a potential means of treating acid-related disorders in patients who are unable or unwilling to swallow intact capsules or tablets of PPIs, which may include for example omeprazole, lansoprazole, pantoprazole, rabeprazole, esomeprazole and leminoprazole. Such patients may be unwilling or unable to swallow intact capsules or tablets due to any medical or neurological condition leading to dysphagia, unconsciousness, coma, critical illness or severe illness of any sort.

### DETAILED DESCRIPTION OF THE PREFERRED EMBODIMENT

The purpose of the present invention is to provide a formulation and composition of a pharmaceutical for treatment of gastrointestinal conditions or acid-related disorders in patients who are unable or unwilling to swallow intact capsules or tablets of PPIs. The inability or unwillingness to swallow intact capsules or tablets may be due to a medical or neurological condition leading to dysphagia, inconsciousness, coma, critical illness or severe illness of any sort. The above-noted pharmaceutical composition may include an aqueous suspension of an acid-labile drug, such as substituted benzimidazoles/PPIs in a pharmaceutically acceptable carrier. The benzimidazoles are considered to be chemically converted in the body to become pharmacologically active, which implies that they are pro-drugs.

Alternatively, a pharmaceutical composition of a solid mixture of an acid-labile drug is also taught and may include but is not limited to benzimidazoles/PPIs having a solid-phase basic salt of a metal formulated as a capsule, a standard tablet or an effervescent tablet. These PPI materials may be introduced to the patient through a plurality of modes such as standard tablets, effervescent tablets, capsules, as suspensions in an acceptable carrier, or as fine-grained suspensions in a carrier for intravenous communication to a patient. The introduction of the fine-grained PPI in a calcium solution should obviate clumping for introduction of the PPI to the patient who is unable or unwilling to swallow.

Pancreatic enzymes are presently formulated as encapsulated enteric-coated or non-coated granules, which coated granules may be pancrease™, pancreolipase™, or others, and the non-coated granules may be Viokase™ or Kotazyme™, for example. The enteric-coated granules protect the molecules of pancreatic enzymes, which are proteins, from the destructive effects of gastric acid and allow them to be released in the small intestine. Non enteric-coated preparations of pancreatic enzymes release the enzymes in the duodenum and proximal small intestine, jejunum, but provide no protection from gastric acid, which reduces the bioavailability of these formulations.

Patients with chronic inflammation and destruction of the pancreas, chronic pancreatitis, typically have severe pain as a debilitating clinical manifestation of their ailment. The release of pancreatic enzyme supplements proximally in the small intestine is known to be critical for treating the pain of chronic pancreatitis. In the duodenum and proximal jejunum, these enzymes inhibit the activation of pancreatic secretion that is triggered by the release of the naturally occurring hormone cholecystokinin, CCK. Therefore, only non-enteric coated formulations of pancreatic enzyme supplements have been shown to be effective in controlling the pain of chronic pancreatitis.

A formulation, such as granules or powder for example, of pancreatic enzyme supplements combined in a capsule with a basic salt could improve bioavailability of the pancreatic enzyme supplement, by neutralizing gastric acid. Alternatively, the formulation of pancreatic enzyme supplement can be combined in a tablet or liquid formulation to improve bioavailability of the pancreatic enzyme supplement. This should also allow the pancreatic enzyme supplement to be released in the duodenum and proximal jejunum where, by inhibiting CCK-stimulated pancreatic secretion, the enzymes can be effective in preventing the attacks of pain from chronic pancreatitis.

The present invention allows administration of pancreatic enzyme supplements in tablet or liquid form. These enzyme supplement forms or formulations have antacid properties required to prevent the degradation or destruction of pancreatic enzyme molecules by acid present in the stomach and duodenum. Prevention of enzyme supplement degradation allows patients an alternative means of taking pancreatic enzyme supplements. For patients unable or unwilling to swallow intact capsules or tablets, prevention of the degradation of the enzyme supplements also allows continued administration of pancreatic enzyme supplements to patients requiring them for a medical indication. Similarly, the substituted benzimidazoles may be provided in a tablet or liquid form, although they may be provided as particulates dissolved or suspended in solution.

The above-described forms and formulations for the administration of the pharmaceutical also apply to any enteric-coated preparations of acid-labile pharmaceuticals protected against destruction, degradation or chemical alteration by the effects of acid within the stomach or elsewhere in the upper gastrointestinal tract, which are approved for medical use at a later date.

The below-noted metallic salts in solution or suspension include, but are not limited to the carbonates, bicarbonates and hydroxides of sodium, potassium, calcium, magnesium and aluminum. The enteric-coated pharmaceutical compounds include, but are not limited to pancreatic enzyme supplements and substituted benzimidazoles/proton pump inhibitors, which PPIs include, but are not limited to omeprazole, lansoprazole, pantoprazole, rabeprazole, esomeprazole and leminoprazole. The previously discussed artificial tubes inserted into the gastrointestinal tract include for example, but are not limited to nasogastric, nasoduodenal, nasojejunal, orogastric, oroduodenal, orojejunal, gastrostomy and jejunostomy tubes. Gastrostomy and jejunostomy tubes may be surgically, radiologically or endoscopically placed in a patient.

A pharmaceutical preparation of a solution or suspension of a metallic salt having a solution or suspension pH>7.0 is provided for administration of the pharmaceutical preparation to patients. More specifically, an enteric-coated pharmaceutical compound can be suspended in the noted solution or suspension for subsequent administration to human beings or other animals, either orally or through an artificial tube inserted into the gastrointestinal tract. A pharmaceutical preparation may also be prepared from a solid mixture of the metallic salt with a solution or suspension pH>7.0, and the acid-labile pharmaceutical compound is then available for subsequent administration to human beings or other animals, either orally as a capsule or tablet whether regular or effervescent. The noted solid mixture may be suspended in water by opening the capsule or dissolving and suspending the tablet for communication through an artificial tube inserted into the gastrointestinal tract for an alternative administration of the pharmaceutical preparation.

The pharmaceutical preparations can be used for the treatment of appropriate conditions consistent with good medical or veterinary practice and appropriately dosed for the noted condition. Exemplary conditions requiring administration of a PPI in this manner include, but are not limited, to the following: GERD, peptic ulcer of the duodenum or stomach, gastritis, H. pylori infection, pathological hypersecretion of gastric acid due to Zollinger-Ellison syndrome or other pathological conditions, the prevention or treatment of bleeding peptic ulcer of the stomach or duodenum, and the prevention or treatment of stress-related gastric mucosal disease in critically ill patients or in any patient in an ICU. Similar conditions requiring administration of pancreatic enzymes in this manner include, but are not limited to, acute and chronic pancreatitis and their complications or sequelae.

The formulations of the present invention can be administered in various ways, as noted above. These formulations could be manufactured in a concentrated form, such as a capsule, a standard tablet or an effervescent tablet, which are only examples and not limitations. Thereafter, the formulations are available for routine administration, as oral application, as a suspension in a fluid of through communication through a tube.

Granules of omeprazole and lansoprazole and tablets of pantoprazole and rabeprazole have been suspended in calcium carbonate, magnesium hydroxide and aluminum hydroxide suspension. A suspension produced in this manner is suitable for oral administration to human beings.

More specifically, suspensions of calcium carbonate were produced by dissolving either 400 mg or 800 mg of calcium carbonate in 10 cc of water, resulting in suspensions of 4 gm % and 8 gm %, respectively. In separate 10 cc preparations of either strength of the calcium carbonate suspension, the granular contents of a 20 mg omeprazole capsule, a 30 mg lansoprazole capsule, a 20 mg rabeprazole tablet, and a 40 mg pantoprazole tablet were suspended. In approximately 30 minutes, the granules of the omeprazole and lansoprazole capsules were in complete suspension. In approximately two hours, the contents of the tablets of rabeprazole and pantoprazole had completely suspended.

Similarly, the granular contents of a 20 mg omeprazole capsule, a 30 mg lansoprazole capsule, a 20 mg rabeprazole tablet and a 40 mg pantoprazole tablet have been suspended in separate suspensions of aluminum hydroxide 400 mg per 10 cc of water and magnesium hydroxide 400 mg per 10 cc of water.

An alternative fine-mesh powder form of these formulations can be obtained by crushing PPI granules or tablets with calcium carbonate, magnesium hydroxide or aluminum hydroxide. The requisite formulation can be provided by mixing the active pharmaceutical compound with any of the calcium carbonate, magnesium hydroxide or aluminum hydroxide salts, which mixtures can be formulated either as a capsule, a standard tablet or an effervescent tablet.

A PPI of the present invention may be administered to patients either by mouth or by an artificial tube inserted into the gastrointestinal tract. The latter tube methods include but are not limited to nasogastric, nasoduodenal, nasojejunal, orogastric, oroduodenal, orojejunal, gastrostomy and jejunostomy tubes. The gastrostomy and jejunostomy tubes may have been surgically, radiologically or endoscopically placed in the patient. An example of a patient user may be a conscious patient with less than complete dysphagia. Further examples of potential patient-users may be conscious patients who do not want to swallow an intact capsule or tablet, or patients who subjectively feel that they cannot swallow an intact capsule or tablet despite objective evidence of otherwise normal swallowing functions.

The PPI can be administered in a small total volume of a liquid that has intrinsic antacid properties, such as calcium carbonate, magnesium hydroxide or aluminum hydroxide, for example. This liquid will further assist in reducing the total volume of acidic juice within the stomach and upper gastrointestinal tract by simple chemical neutralization. In GERD, this may have the clinical advantage of supplying immediate symptom relief from the antacid properties of the solution or suspension and subsequent sustained relief through the pharmacological action of the absorbed PPI once it inhibited the membrane-bound molecules of the proton pump.

The use of an aqueous calcium solution or suspension, which may be calcium carbonate for example, might also provide an additional small physiological stimulus to gastric acid secretion by the parietal cell mass. PPI's are required to be taken up by parietal cells before they can exert their pharmacological action on actively secreting membrane-bound molecules of H+/K+-ATPase. Thus, it is expected that the calcium solution or suspension would enhance the pharmacological effectiveness of the PPI as these drugs are more likely to be taken up by parietal cells that are, at least temporarily, stimulated into secreting acid through the activation of membrane-bound molecules of H+/K+-ATPase, the proton pump, by the activity of the calcium in the liquid formulation ingested or otherwise administered.

In addition, the regular use of a PPI or other acid-labile drug in a solution or suspension of a calcium salt, such as calcium carbonate, should help the individual attain his or her recommended daily intake of calcium important in the prevention of age-related or post-menopausal loss of bone mass, which is commonly referred to as osteoporosis.

### REFERENCES

Andersson et al., Pharmacokinetics and bioavailability of omeprazole after single and repeated oral administration in healthy subjects. British Journal of Clinical Pharmacology 1990; 29: 557-563.

Andersson et al., Pharmacokinetics of various single intravenous and oral doses of omeprazole. European Journal of Clinical Pharmacology 1990; 39: 195-197.

Andersson et al., Pharmacokinetics of [14C]omeprazole in patients with liver cirrhosis. Clinical Pharmacokinetics 1993; 24: 71-78.

Chun et al., Lansoprazole: An alternative method of administration of a capsule dosage formulation. Clinical Therapeutics 1995; 17: 441-447.

Chun et al., Lansoprazole: Administration of the contents of a capsule dosage formulation through a nasogastric tube. Clinical Therapeutics 1996; 18: 833-842.

Landahl et al., Pharmacokinetic study of omeprazole in elderly healthy volunteers. Clinical Pharmacokinetics 1992; 23: 469-476.

Lochart S P, Baxter G. A lansoprazole suspension formulation as an alternative to capsules for oral administration. Digestion 1998; 59: 226 (Abstract)

Phillips et al., A prospective study of simplified omeprazole suspension for the prophylaxis of stress-related mucosal damage. Critical Care Medicine 1996; 24: 1793-1800.

Pilbrant et al., Development of an oral formulation of omeprazole. Gastroenterology 1985; 108: 113-120

Regardh et al., The pharmacokinetics of omeprazole in humans—a study of single and intravenous and oral doses. Therapeutic Drug Monitoring 1990; 12: 163-172.

Sharma et al., Nonencapsulated, intact omeprazole granules effectively inhibit intragastric acidity when administered via a gastrostomy. American Journal of Gastroenterology 1997; 92: 848-851.

Sharma et al., The pharmacodynamics of lansoprazole administered via gastrostomy as intact, nonencapsulated granules. Alimentary Pharmacology and Therapeutics 1998; 12: 1171-1174.

Sharma et al., The effects on intragastric acidity of per-gastrostomy administration of an alkaline suspension of omeprazole. Alimentary Pharmacology and Therapeutics 1999a; 13: 1091-1095.

Sharma et al., Simplified lansoprazole suspension—a liquid formulation of lansoprazole—effectively suppresses intragastric acidity when administered through a gastrostomy. American Journal of Gastroenterology 1999b; 94: 1813-1817.

Sharma et al., Oral pharmacokinetics of omeprazole and lansoprazole after single and repeated doses as intact capsules and as suspensions in sodium bicarbonate. Alimentary Pharmacology and Therapeutics 2000a; 14: 887-892.

Sharma et al., Simplified lansoprazole suspension is as effective as intact lansoprazole capsule in reducing gastric acid secretion. American Journal of Gastroenterology 2000b; 95: in press (Abstract).

Tolman et al., The effects of oral doses of lansoprazole and omeprazole on gastric pH. Journal of Clinical Gastroenterology 1997; 24: 65-70.

From the foregoing detailed description, it can thus be seen that the present invention provides an improved method for the formulation and delivery for administration of acid-labile drugs to human beings and other animals. The present method is achieved by mixing the active pharmaceutical compound with a basic salt in a solution or suspension of any kind, where the basic salt solution or suspension protects the pharmaceutical compound from the adverse effects of gastric acid by neutralizing gastric acid.

While there has been illustrated and described what is at present considered to be a preferred embodiment of the present invention, it will be understood by those skilled in the art that various changes and modifications may be made, and equivalents may be substituted for elements thereof without departing from the true scope of the invention. In addition, many modifications may be made to adapt a particular situation or material to the teachings of the invention without departing from the central scope thereof. Therefore, it is intended that this invention not be limited to the particular embodiment disclosed as the best mode contemplated for carrying out the invention, but that the invention will include all embodiments falling within the scope of the appended claims.

What is claimed is:

1. A method for the formulation and delivery of an acid-labile pharmaceutical compound from among at least the group of substituted benzimidazoles and pancreatic enzyme supplements, said method comprising:
a. providing an active pharmaceutical compound;
b. providing a basic salt as one of a suspension and a solution;
c. combining said pharmaceutical compound in a form as one of a tablet, a capsule, a powder, a solution and a suspension to provide said acid-labile pharmaceutical compound.

2. A method for the formulation and delivery of an acid-labile pharmaceutical compound from among at least the group of substituted benzimidazoles and pancreatic enzyme supplements as claimed in claim 1, wherein said one of said basic salt solution and suspension has a pH greater than 7.0.

3. A method for the formulation and delivery of an acid-labile pharmaceutical compound from among at least the group of substituted benzimidazoles and pancreatic enzyme supplements as claimed in claim 2, wherein said basic salt is one of a Type IA and Type II metal salt.

4. A method for the formulation and delivery of an acid-labile pharmaceutical compound from among at least the group of substituted benzimidazoles and pancreatic enzyme supplements as claimed in claim 3, wherein said metal salt is one of sodium, potassium, magnesium, calcium and aluminum salt.

5. A method for the formulation and delivery of an acid-labile pharmaceutical compound from among at least the group of substituted benzimidazoles and pancreatic enzyme supplements as claimed in claim 1, wherein said compound in said formulation includes a therapeutic dose of said pharmaceutical compound.

6. A method for the formulation and delivery of an acid-labile pharmaceutical compound as claimed in claim 1, further providing an enteric-coated formulation for said pharmaceutical compound formulation; and,
combining said enteric-coated formulation by one of mixing, dissolving and suspending with said one of said basic salt, basic salt solution and basic salt suspension to provide said acid-labile pharmaceutical compound.

7. An acid-labile pharmaceutical compound having at least substituted benzimidazoles and pancreatic enzyme supplements, said acid-labile pharmaceutical compound comprising:
a. an active pharmaceutical compound;
b. a basic salt, which basic salt is at least one of a solution and suspension;
c. said one of said solution and suspension having a pH greater than 7.0;
d. said active pharmaceutical compound and said basic salt combined as at least one of a form of a tablet, capsule, and powder; and
e. said at least one of a form of a tablet, capsule and powder provided to said one of a solution and suspension to provide said acid-labile pharmaceutical compound operable to provide at least one of neutralization of gastric acid and temporary stimulation of gastric acid secretion.

8. An acid-labile pharmaceutical compound as claimed in claim 7, wherein said basic salt is one of a Type I and Type IIA metal.

9. An acid-labile pharmaceutical compound as claimed in claim 8, wherein said metal is one of sodium, potassium, magnesium, calcium and aluminum.

10. An acid-labile pharmaceutical compound as claimed in claim 7, wherein said pharmaceutical compound in said at least one form has at least a therapeutic dose of said pharmaceutical compound.

11. An acid-labile pharmaceutical compound as claimed in claim 10, wherein said at least one form of said pharmaceutical compound has said basic salt in each said therapeutic dose, said basic salt having a concentration between about 1 mM and about 1M per therapeutic dose.

12. An acid-labile pharmaceutical compound as claimed in claim 7, wherein said compound may be dispensed in said form of a tablet, capsule, powder, solution and suspension.

13. An acid-labile pharmaceutical compound as claimed in claim 7, wherein said compound may be administered into a gastrointestinal tract by at least one of orally and by artificial tube, said tube being at least one of nasogastric tube, nasoduodenal tube, hasojejunal tube, oragastric tube, oraduodenal tube orajejunal tube, gastrotomy tube and jejunostomy tube.

14. An acid-labile pharmaceutical compound as claimed in claim 13, wherein said gastrotomy tube and jejunostomy tube may be provided by at least one of surgical, radiological and endoscopical means.

15. An acid-labile pharmaceutical compound as claimed in claim 13, wherein said compound is provided and mixed in one of said solution and suspension for introduction to a patient through said tube.

16. A method for the formulation and delivery of an acid-labile pharmaceutical compound as claimed in claim 1, wherein said benzimidazole compound is one of omeprazole, lansoprazole, pantoprazole, rabeprazole and esomeprazole.
